(19) United States
(12) Patent Application Publication
(10) Pub. No.: US 2020/0155634 A1
(43) Pub. Date: May 21, 2020

(54) COSMETIC COMPOSITION COMPRISING COLEUS FORSKOHLII AND CASSIA OCCIDENTALIS AND/OR CASSIA ALATA, AND COMPOSITIONS FOR USE IN TREATING VITILIGO

(71) Applicant: Life Science Investments Ltd, London (GB)

(72) Inventors: Silvia CHAMI DE DIEHL, London (GB); Christian DIEHL, London (GB)

(73) Assignee: Life Science Investments Ltd, London (GB)

(21) Appl. No.: 16/630,904

(22) PCT Filed: Jul. 16, 2018
(86) PCT No.: PCT/IB2018/055248

§ 371 Date: Jan. 14, 2020

Related U.S. Application Data
(63) Continuation-in-part of application No. PCT/IB2017/054256, filed on Jul. 14, 2017.

(51) Int. Cl. A61K 36/482; A61K 8/9789; A61K 36/53; A61P 17/00; A61Q 19/04; A61Q 5/00

(52) U.S. Cl. CPC A61K 36/482; A61K 8/9789; A61Q 5/00; A61P 17/00; A61Q 19/04; A61K 36/53

(57) ABSTRACT

A topical composition comprising:
a) a cosmeceutically acceptable vehicle;
b) an extract of Coleus forskohlii; and
c) an extract of Cassia occidentalis and/or Cassia alata.

### TECHNICAL FIELD

The present invention relates to a cosmeceutical composition comprising plant extracts. The invention further relates to such a composition for therapeutic or non-therapeutic use to treat dermatological infections related to a depigmentation phenomenon and/or canities. The composition according to the invention is also used as a tanning composition.

### BACKGROUND

The skin has epidermal units that are responsible for melanin production and distribution, a process called melanogenesis. These units, named epidermal melanin unit (EMU) are composed of a melanocyte surrounded by keratinocytes and regulated by a closed paracrine system.

Melanin is the primary determinant of skin, hair, and eye colour. Besides defining an important human phenotypic trait, it has a critical role in photoprotection due to its ability to absorb ultraviolet radiation (UV). Constitutive pigmentation reflects the genetically determined level of melanin and can be changed by several regulatory factors. These factors may be intrinsic (released by keratinocytes and fibroblasts, endocrine, inflammatory and neuronal cells) or extrinsic (UV and drugs).

Melanogenesis is a complex process with different stages. When disturbed, it may determine different types of pigmentation defects, which are classified as hyperpigmentation or hypopigmentation (or depigmentation) and which may occur with or without an altered number of melanocytes. The understanding of the mechanisms of melanogenesis helps to explain the pigmentation defects observed in hypopigmentation or hyperpigmentation, notably in diseases as pytiriasis or vitiligo, and allows the development of potential therapeutic strategies.

Melanocytes are specific cells located at the bottom layer of the epidermis. They produce the melanin, namely the skin pigment, through the biochemical pathway of melanogenesis.

In mammalians including humans, melanocytes reside in the basal layer of epidermis where they form the EMU. The ratio of melanocytes to keratinocytes is 1:10 in the epidermal basal layer of the epidermis. About 1200 melanocytes exist per mm² of the skin independently of the human race.

In the hairy region of mammalian skin, melanoblasts (precursor cells of a melanocytes) further enter the newly developing hair follicles where they finally become localized. Once in the hair follicles, melanoblasts are segregated into two populations: one consists of hair matrix melanocytes, which are responsible for pigmentation of the initial hairs; the other population consists of melanocyte stem cells, which are localized at the lower permanent portion of the hair follicle (the bulge region) and are responsible for the maintenance of the hair follicle pigmentary system in the subsequent hair cycles. In non-hairy regions of the human skin, melanoblasts stay immature and reside on the basement membrane of the epidermis where they undergo terminal differentiation into mature melanocytes upon stimulation from keratinocytes.

Follicular pigmentation is a result of structural and functional interactions between follicular melanocytes, matrix keratinocytes and dermal papilla fibroblasts. This tripartite system is described as the hair melanin unit or follicular melanin unit. The process of hair pigmentation includes the melanogenic activity of follicular melanocytes, the transfer of melanin granules into keratinocytes and the formation of pigmented hair shafts. It is considered that a transport of melanin granules to keratinocytes in the growing hair shaft is similar to the epidermal phagocytosis of melanosomes mediated by receptor PAR2 on keratinocytes.

Melanogenesis is primarily regulated by α-melanocyte-stimulating hormone (α-MSH) which binds to the Melanocortin-1 receptor (MC1R) to activate it, leading to the activation of the cAMP pathway, and in fine activation of the cytoplasmic protein kinase A (PKA). This increased PKA activity leads to the increased activity of tyrosinase, dendrite formation and proliferation of melanocytes.

The melanogenic effects of α-MSH can be advantageously mimicked by compounds stimulating the cAMP pathway in melanocytes.

Two types of melanin are produced by human melanocytes: eumelanin (black) and phaeomelanin (yellow/reddish), their balance depending on individual genetic predispositions and provide the phototype of the skin or of the hair color.

The biochemical process of melanogenesis is as follows: tyrosinase (TYR) carries out tyrosine hydroxylation to L-3,4-dihydroxyphenylalanine (DOPA) which is rapidly oxidized to DOPAquinone. In the presence of cysteine DOPAquinone reacts with it, yielding 3- or 5-cysteinylDOPAs, which then oxidize and polymerize, giving rise to yellow-red soluble melanin_phaeomelanin. In the absence of thiols (cysteine, glutathione or thioredoxin) brown-black eumelanin is produced. DOPAquinone spontaneously undergoes cyclization to DOPAchrome. The DOPAchrome spontaneously loses carboxylic acid and generates 5,6-dihydroxyindole (DHI), which rapidly oxidizes and polymerizes to form dark brown-black, insoluble DHI-melanin. However, if DOPAchrome tautomerase (TYRP2/DCT) is present, DOPAchrome will form DHI-2-carboxylic acid (DHICA). Tyrosinase and TYRP1 catalyse further conversions obtaining finally a lighter brown colour DHICA-melanin.

Besides its role of the skin pigment, melanin protects the body by absorbing ultraviolet radiation. Excessive UV radiation causes sunburn along with other direct and indirect DNA damage to the skin, and the body naturally combats and seeks to repair the damage and protect the skin by creating and releasing further melanin into the skin's cells. With the production of the melanin, the skin color darkens. This process corresponds to the the tanning process. Thus, MC1R has been known to be implicated in different UV-induced reaction such as pigmentation and adaptive tanning. UV-induced tans act as a photoprotection by providing a sun protection factor (SPF) of 3-4 and epidermal hyperplasia.

Graying of the hair is an important cause of low self-esteem, often interfering with socio-cultural adjustment. The onset and progression of graying of the hair also designated as canities correlate very closely with chronological aging, and occur in varying degrees in all individuals eventually, regardless of gender or race. Canities, such as premature canities, may occur alone as an autosomal dominant condition or in association with various autoimmune or premature aging syndromes. Reduction in melanogenically active melanocytes in the hair bulb of gray anagen hair follicles with resultant pigment loss is central to the pathogenesis of graying. Defective melanosomal transfers to cortical keratinocytes and melanin incontinence due to melanocyte degeneration are also believed to contribute to this.

Despite the extensive molecular research being carried out to understand the pathogenesis of canities, there is paucity of effective evidence-based treatment options.

An alternative to the treatment of canitie is hair dyes which are commonly used to dye grayed hair. The use of such dyes is troublesome and sometimes causes side-effects such as rash of the scalp. Therefore, many users find hair dyes to be an unsatisfactory solution of the graying of the hair.

Under such circumstances, it has been desired to develop a pharmaceutical preparation or a cosmetic composition for application to hair capable of essentially preventing graying of the hair and/or restoring grayed hair to its natural color.

In a same manner, the many types of skin pigmentation disorders may present in diverse forms and distributions and have various causes. They can be inherited (eg, vitiligo, familial periorbital hyperpigmentation), acquired (eg, postinflammatory pityriasis alba, idiopathic guttate hypomelanosis, Becker's nevus, melasma), infectious (eg, tinea versicolor), benign and self-limiting (eg, isolated café au lait spots, photocontact dermatitis), or a sign of more serious underlying disease (eg, multiple café au lait spots, malignant acanthosis nigricans). As example, pityriasis alba usually presents as ill defined, scaly patches of hypomelanosis on the cheeks of people with an atopic diathesis. The face is also a favored site for vitiligo, but the distribution is periorificial, and the pigment loss is complete because of a destruction of melanocytes.

In particular, vitiligo is an autoimmune disease featuring a progressive loss of pigmentation of the skin. In vitiligo, this loss of pigmentation is directly correlated with a loss of melanocytes, resulting at the onset of the disease in white patches of different sizes appearing on different parts of the body.

It is reported in the literature that about 1% of the world's population has vitiligo. Vitiligo affects both genders equally, although is a common observation that women complain earlier and more frequently about vitiligo, possibly because in some places vitiligo is considered as a stigma or a cosmetic problem.

Although the aetiology of vitiligo remains unclear, several theories have been developed. However, the autoimmune hypothesis remains the leading one, involving immune attacks of melanocytes by both T and B cell dependent mechanisms. The theory of autoimmune mediated destruction of melanocytes is well accepted and seems to have currently become the leading hypothesis in vitiligo pathogenesis.

The immune reaction can be mediated by cellular immunity, humoral antibody mediated immunity, and the action of cytokines. Cell-mediated immunity in vitiligo is demonstrated by the presence of inflammatory infiltrates in perilesional vitiligo skin. Decreased CD4+ to CD8+ lymphocytes ratio in vitiligo-stricken skin compared to healthy skin and CD8 T cells directed against melanocytic antigens have been found both in perilesional skin and in the blood of vitiligo patients. This shows that the elimination of melanocytes by cytotoxic T cells is a mechanism leading to depigmentation in vitiligo.

Cytokines also seem to play an important role in vitiligo pathogenesis. There is an increase in the expression of tumour necrosis alpha (TNF-α) and interferon-gamma (IFN-γ), suggesting that vitiligo is mediated by a T helper cell-1 (Th1) response.

There is also probably an inflammatory component in vitiligo as the levels of various pro-inflammatory cytokines are increased in vitiligo, especially Interleucin-1α (IL-1α) and Interleucin-6 (IL-6). Interestingly, it was found that IL1α, TNF-α, and IL-6 elicited a dose-dependent decrease in the activity of the enzyme tyrosinase of cultured normal human melanocytes and also inhibited melanocyte proliferation.

On the other hand, oxidative stress is considered to be one of the possible pathogenic events in melanocyte loss. Studies showed systemic oxidative stress in patients with vitiligo due to an imbalance in enzymatic and non-enzymatic antioxidant systems. The intracellular levels of H₂O₂ and other reactive oxygen species (ROS) also increase in response to cytokines such as TNF-α and transforming growth factor β1 (TGF-β1), which are potent inhibitors of melanogenesis.

Depigmentation disease recovery depends on a viable melanocyte reservoir and in many patients with skin disease related to depigmentation, as vitiligo, repigmentation is possible when pigment cells are stimulated with appropriate topical or oral medications.

Melanocytes for repigmentation by medical methods arise from three main sources: (a) the hair follicle unit; (b) unaffected melanocytes within areas of depigmented epidermis, and (c) melanocytes located at the edge of skin depigmentation lesions. Most melanocytes originate from the hair follicle unit where they are present in large numbers and migrate towards the epidermis. A striking feature of the hair follicle reservoir is the enormous potential for providing pigment cells considering its tiny size and diameter.

Besides, hair follicles are absent on palms, soles, mucosal or semi-mucosal surfaces and therefore these areas become particularly refractory to all therapies because the melanocyte reservoir is lacking. In other words, an essential need to treat skin disease related to depigmentation is to improve the migration of melanocyte to damaged skin area.

As skin infection related to depigmentation causes little or no direct physical impairment, it is often considered just as a cosmetic problem. However, the change in appearance caused by this skin disorder can affect a person's emotional and psychological well-being, having major consequences on his/her life. Moreover, skin infection related to depigmentation could be a long-lasting disease, and its unpredictable natural course causes a heavy burden on patients' quality of life (QoL).

The Applicant has developed a novel composition to solve the issues described above.

### DESCRIPTION OF THE INVENTION

For repigmentation to occur, it is necessary that melanocytes become stimulated with appropriate signals. In this regard, two important properties of melanocytes have to be taken into consideration: (a) neo-melanogenesis, which implies melanin synthesis and production of melanosomes and b) melanocyte migration, which will help pigment cells to reach depigmented skin.

Surprisingly, the Applicant has highlighted that a specific combination of vegetal extracts allows to meets the specific needs above mentioned (treatment of skin diseases related to depigmentation or canities, tanning agent, increase of melanogenesis and melanocyte migration . . . ).

According to the present invention, the terms “plant extract(s)” and “vegetal extract(s)” are equally used to refer to a substance or an active with desirable properties that is removed from the tissue of a plant, usually by treating it with a solvent.

The present invention relates to a composition, advantageously a cosmetic composition, comprising a combination of vegetal extracts, from the plants Coleus forskohlii, Cassia occidentalis and/or Cassia alata.

Compositions are disclosed for cosmeceuticals that aid in the retardation of the progression of canities or skin disease related to depigmentation such as vitiligo and/or at least partial repigmentation of the white patches.

More specifically, the compositions herein disclosed are based on the use of a combination of active vegetal extracts, from the plants Coleus forskohlii, Cassia occidentalis and Cassia alata. These compositions are suitable for stimulating the survival, proliferation and differentiation of melanocytes, and activating melanogenesis. These active ingredients also possess antioxidant and anti-inflammatory activities, may stimulate the cAMP pathway and increase the expression of MC1R.

In one aspect of the invention, the composition contains a cosmeceutically effective amount of both extracts of Coleus forskohlii, Cassia occidentalis and/or Cassia alata, in a cosmeceutically acceptable vehicle.

The composition according to the invention is a topical composition comprising:
a) a cosmeceutically acceptable vehicle;
b) an extract of Coleus forskohlii; and
c) an extract of Cassia occidentalis and/or Cassia alata.

Indeed, the present invention is based on the discovery that the combination of vegetal extracts, namely Coleus forskohlii, Cassia occidentalis and/or Cassia alata, increases the number and activity of melanocytes in a more effective manner than each extract taken alone. In a second step, it has been discovered that the use of this combination of plant extracts in a cosmeceutically acceptable vehicle was an effective treatment for skin disease related to depigmentation, such as for example vitiligo, when applied topically on lesions of this disease.

Desired result may include to increasing the expression of Stem Cell Factor (SCF) and Basic Fibroblast Growth Factor (bFGF), and/or stimulating the conversion of melanoblasts to active melanocytes, and/or promoting the number of keratinocytes and stimulate their activity, and/or activating cAMP pathway and negatively reducing T-cell proliferation, and/or stimulating the Wnt/f3 catenin pathway, and/or reducing the levels of Transforming Growth Factor-beta (TGF-β), and/or reducing the levels of interferon-gamma (IFN-γ), and/or reducing the levels of Tumour Necrosis Factor-alpha (TNF-α), and/or reducing the levels of various pro-inflammatory cytokines, especially IL-la and IL-6, and/or reducing the levels of oxidative stress, and/or inhibiting Nitric Oxide Synthase (NOS) and nitric oxide (NO) production, and/or increasing the expression of MC1R, and/or restoring the physiological levels of Microphtalmia Transcription Factor (Mitf), increasing melanocyte production, or a combination of these effects whose aim is the repigmentation of white patches of the skin. Cosmeceutical compositions may refer to, but are not limited to, for example cosmetics, drugs, therapeutics, pharmaceuticals, medicaments, medications, medicines, remedies.

“Cosmeceutically acceptable” and “cosmeceutically effective” refer to compositions that bring out the desired result.

According to a particular embodiment, the composition according to the invention further comprises a penetration enhancer, advantageously a skin penetration enhancer.

According to a particular embodiment, the extract of Coleus forskohlii is a root extract, advantageously an ethanol/propylene glycol root extract; the extract of Cassia occidentalis is a pod extract, advantageously a pod methanolic extract; the extract of Cassia alata is a leave extract, advantageously a methanolic leave extract.

According to an embodiment, the Coleus forskohlii extract represents between 0.01 and 15.0% in weight of the composition, advantageously between 0.1 and 10.0%, preferably between 5.0 and 8.0%.

According to an embodiment, the Cassia occidentalis or Cassia alata extract represents between 0.01 and 10.0% in weight of the composition, advantageously between 0.1 and 8.0%, preferably between 2.0 and 5.0%.

According to a particular embodiment, the weight ratio of Coleus forskohlii extract:Cassia occidentalis and/or Cassia alata represents 3:1.

According to an embodiment, the penetration enhancer is diethylene glycol monoethyl ether and represents between 0.5 and 5.0% in weight of the composition, advantageously between 1.0 to 2.0%.

According to an embodiment, the pH of the composition according to the invention is between 6.5 to 7.1.

According to a particular embodiment, the composition has a specific pH of 6.8 which appears to be the most convenient value for the melanosomes activity.

According to an embodiment, the composition contains a buffer which permits to reach a pH as close as possible to 6.8

According to an embodiment, the composition contains a cosmeceutically acceptable preservative.

According to a particular embodiment, the composition is a leave-on product or a rinse-off product.

According to another aspect of the invention, the composition above described is for therapeutic or non-therapeutic use to promote the proliferation of melanocytes in the skin and/or in the hair follicle unit.

According to the invention, the composition is for therapeutic or non-therapeutic use to promote the melanogenesis in the skin and/or in the hair follicle unit.

According to the invention, the composition is for therapeutic or non-therapeutic use to treat canitie.

According to another aspect of the invention, the composition above described is for therapeutic or non-therapeutic use as a tanning agent.

According to another aspect, the composition according to the invention is for therapeutic or non-therapeutic use to treat skin disease related to depigmentation in a human subject.

According to an embodiment, the skin disease related to depigmentation is selected from the group consisting of pytiriasis alba, pytiriasis versicolor, idiopathic guttate hypomelanosis, progressive macular hypomelanosis, post-inflammatory hypopigmentation and vitiligo.

According to a particular embodiment, the skin disease related to depigmentation is vitiligo.

According to the invention, the composition contains a cosmeceutically effective amount of a combination of an extract of Coleus forskohlii, an extract of Cassia occidentalis and/or an extract of Cassia alata, a penetration enhancer, a buffer and a preservative in a cosmeceutically acceptable medium.

In another aspect, the composition according to the invention containing a cosmeceutically effective amount of a combination of an extract of Coleus forskohlii, an extract of Cassia occidentalis and/or an extract of Cassia alata, a penetration enhancer, a buffer and a preservative in a cosmeceutically acceptable medium is topically applied.

In another aspect, the composition according to the invention contains a cosmeceutically effective amount of a combination of an extract of Coleus forskohlii, an extract of Cassia occidentalis and/or an extract of Cassia alata, a penetration enhancer, a buffer and a preservative in a cosmeceutically acceptable medium treats skin disease related to depigmentation, advantageously selected from the group consisting of pytiriasis alba, pytiriasis versicolor, idiopathic guttate hypomelanosis, progressive macular hypomelanosis, post-inflammatory hypopigmentation and vitiligo, preferably vitiligo.

In one aspect, the composition acts to increase the expression of Stem Cell Factor (SCF) and Basic Fibroblast Growth Factor (bFGF).

In another aspect, the composition acts to stimulate the conversion of melanoblasts to active melanocytes and hence promote the proliferation of melanocytes.

In another aspect, the composition acts to promote the number of keratinocytes and stimulate their activity.

In another aspect, the composition acts to activate cAMP pathway and negatively reduce T-cell proliferation.

In another aspect, the composition acts to stimulate the Wnt/B catenin pathway.

In another aspect, the composition acts to reduce the levels of Transforming Growth Factor-beta (TGF-β).

In another aspect, the composition acts to reduce the levels of interferon-gamma (IFN-7).

In another aspect, the composition acts to reduce the levels of Tumour Necrosis Factor-alpha (TNF-α).

In another aspect, the composition acts to reduce the levels of various pro-inflammatory cytokines, especially IL-la and IL-6.

In another aspect, the composition acts to reduce the levels of oxidative stress.

In another aspect, the composition acts to inhibit Nitric Oxide Synthase (NOS) and nitric oxide (NO) production.

In another way, the composition acts to increase the expression of MC1R.

In another aspect, the composition acts to restore the physiological levels of Microphtalmia Transcription Factor (Mitf).

Coleus forskohlii extracts increase the expression of Stem Cell Factor (SCF) and Basic Fibroblast Growth Factor (bFGF). Cassia occidentalis and/or Cassia alata extracts may stimulate the conversion of melanoblasts to active melanocytes.

The Applicant has discovered that the combination of Coleus forskohlii extract and Cassia occidentalis extract may promote melanogenesis in a more important manner than each active ingredient taken separately. In other words, the plant extracts act synergetically.

According to the invention, a composition of the present invention contains from about 5.0 to 8.0% in weight of the composition of a Coleus forskohlii extract (ethanol/propylene glycol root extract), about 2.0 to 5.0% in weight of the composition of a Cassia occidentalis extract (pod methanolic extract) and/or of a Cassia alata extract (leave methanolic extract), about 1.0 to 2.0% in weight of the composition of a penetration enhancer in a buffer solution bringing the composition pH between 6.5 to 7.1, advantageously as close as possible to a value of 6.8, preferably the pH value is 6.8.

According to another aspect, the invention concerns a method of treating skin infection related to depigmentation by topical application to a subject of the composition as above defined.

The invention is further described in detail by reference to the following examples and the attached figures. These examples are provided for purposes of illustration only, and are not intended to be limiting.

The composition of the invention may also be therapeutically or non-therapeutically used in hypomelanotic disorders such as post inflammatory hypomelanosis, infectious or parasitic hypomelanosis (e.g. Pityriasis (Tinea) Versicolor, Leprosy, Treponematoses, Onchocerciasis, Postkala-azar Dermatosis, Herpes Zoster), Halo Nevus, Melanoma associated leukoderma, hypomelanosis from physical agents, hypomelanosis from chemical or pharmacological agents, hypomelanosis of Ito, nevus depigmentosus, Hypopigmented Mycosis Fungoides, Scleroderma and Lichen Sclerosus associated hypomelanosis, Lupus Erythematosus associated hypomelanosis, sarcoidosis associated hypomelanosis and Pityriasis Alba.

In some embodiments, the present invention refers to a composition comprising a cosmeceutically acceptable vehicle. This wording means one or various components of the composition suitable for acting as a diluent, dispersant, or carrier for an active ingredient. In some embodiments, a cosmeceutically acceptable vehicle comprises ingredients commonly used in skin care products such as water, liquid or solid emollients, silicone oils, emulsifiers, solvents, humectants, thickeners and so on. Other ingredients which can be used in the vehicle include penetration enhancers, buffers, preservative agents or moisturizing agents or combinations thereof. Thus, in one embodiment, a cosmeceutically acceptable vehicle suitable for use in the present invention comprises water, propylene glycol dipelargonate, propylene glycol, stearic acid, minerai (paraffinum liquidum) oil, glyceryl stearate, PEG-75 stearate, glycol stearate, cetyl palmitate, avocado (Persea gratissima) oil, triethanolamine, magnesium aluminum silicate, cellulose gum, petrolatum, methylparaben, sorbic acid, xanthan gum. In another embodiment, a cosmeceutically acceptable vehicle comprises water, caprylic/capric triglyceride, glycerine, propylene glycol, decyl oleate, dicaprylyl carbonate, glyceryl stearate, cetearyl alcohol, stearic acid, cetearyl glucoside, xanthan gum, locust bean (Ceratonia siliqua) gum, sodium hydroxide. The cosmeceutically acceptable vehicle may also consist of water, propylene glycol, carbomer, xanthan gum, sorbic acid, disodium EDTA, sodium hydroxide. Other ingredients which may also be used as cosmeceutically acceptable vehicle are water, propylene glycol, carbomer, acrylates/C10-30 alkyl acrylate crosspolymer, sodium hydroxide.

In some embodiments, the present invention comprises a cosmeceutically effective amount of Coleus forskohlii extract. As used herein, the term “cosmeceutically acceptable amount” refers to an amount of Coleus forskohlii extract necessary to achieve a desired result. For example, in some subjects being treated, the cosmeceutically effective amount of Coleus forskohlii extract is dependent on the number of active cells, or area of the skin to be treated. In some embodiments, a composition of the present invention comprises from about 0.01 to 15.0 weight percent Coleus forskohlii extract, from about 0.1 to 10.0 weight percent Coleus forskohlii extract, or about 5 to 8.0 weight percent Coleus forskohlii extract.

In some other embodiments, the composition comprises a cosmeceutically effective amount of Cassia occidentalis extract. As used herein, the term “cosmeceutically acceptable amount” refers to an amount of Cassia occidentalis extract necessary to achieve a desired result.

According to embodiments of the present invention, the composition can be formulated into a number of acceptable forms. For example, in some embodiments a skin care composition can be formulated as aqueous solution, a water-in-oil (w/o) emulsion, an oil-in-water (o/w) emulsion, a dispersion of lipids, an aqueous, water-alcohol, oil or oil-alcohol gel. In some embodiments, if the cosmeceutically acceptable vehicle itself is an (w/o) or (o/w) emulsion, it can contain from about 1 to about 50% of an oil phase and from about 35 to about 95% water, with respect to the weight of the whole composition.

To prepare the topical composition according to the present invention, the usual manner for preparing therapeutic and cosmetic skin care products may be employed.

The active components are generally incorporated in a cosmeceutically acceptable carrier in a conventional manner. The active components can suitably be dissolved or dispersed in a portion of the water or another solvent or liquid to be incorporated in the composition.

In some embodiments, the composition may be in the form of conventional skin-care products such as a cream, gel, lotion or the like. In other embodiments, the compositions of the present invention can be formulated as a “leave-on” product, i.e., a product to be applied to the skin without a deliberate rinsing step after its application to the skin.

The composition may be packaged in any suitable manner such as in a jar, a bottle, an airless bottle or a tube.

In some embodiments, the compositions described in the present invention may be applied one or more times daily to the portion of skin requiring treatment. In some embodiments, the present invention comprises topically applying a composition of the present invention one or more times daily for a period of about 1 to 36 weeks, and beyond. The product is intended to be used long-term.

In one embodiment, a quantity of about 0.25 mg/cm² of a composition of the present invention is applied topically to the skin, spread over and/or rubbed into the skin using the hands or fingers.

In some embodiments, the present invention is directed to a method of treating vitiligo and any over kind of hypopigmentation comprising the step of topically administering to a subject in need thereof, a composition comprising a cosmeceutically acceptable vehicle and a cosmeceutically effective amount of a combination of Coleus forskohlii extract, Cassia occidentalis extract and/or Cassia alata extract.

### FIGURES

FIG. 1 depicts human melanocytes cultured during 96 hours with A/Control (NaCI 0.9% solution) B/Cassia occidentalis extract (5 μg/ml) C/Coleus forskohlii extract (5 μg/ml) and D/Coleus forskohlii extract (5 μg/ml)+Cassia occidentalis extract (5 μg/ml).

FIG. 2 depicts melanin contents of BC16 melanoma murine cells treated during 96 hours in presence of control (NaCI 0.9% solution), Coleus forskohlii extract (C. forskohlii; 5 μg/ml); Cassia occidentalis extract (C. occidentalis; 5 μg/ml); Cassia alata extract (C. alata; 5 μg/ml); the combination of Coleus forskohlii extract (5 μg/ml)+Cassia occidentalis extract (5 μg/ml) or Cassia alata extract (5 μg/ml)±diethylene glycol monoethyl ether (DGEE; 1.5%).

FIG. 3 depicts the values of Microphthalmia-associated transcription factor (Mitf), Tyrosinase-related protein-1 (TRP-1), Tyrosinase-related protein-2 (TRP-2) and Tyrosinase (TYR) in A375 melanocytes after 96 hours in culture with control (NaCI 0.9%), Cassia occidentalis extract (5 μg/ml), Coleus forskohlii extract (5 μg/ml) and combination of Cassia occidentalis extract (5 μg/ml) and Coleus forskohlii (5 μg/ml).

FIG. 4 depicts the levels of Transforming Growth Factor-β in skin biopsies of normal skin and lesional skin of vitiligo patients, and after treatment of the lesional melanocytes with Cassia occidentalis extract (5 μg/ml), Coleus forskohlii extract (5 μg/ml), and the combination of both Cassia occidentalis extract (5 μg/ml) and Coleus forskohlii extract (5 μg/ml).

FIG. 5 depicts the levels of Tumour Necrosis Factor-a in skin biopsies of normal skin and lesional skin of vitiligo patients, and after treatment of the lesional melanocytes with Cassia occidentalis extract (5 μg/ml), Coleus forskohlii extract (5 μg/ml), and the combination of both Cassia occidentalis extract (5 μg/ml) and Coleus forskohlii extract (5 μg/ml).

FIG. 6 depicts the levels of Interferon-γ in skin biopsies of normal skin and lesional skin of vitiligo patients, and after treatment of the lesional melanocytes with Cassia occidentalis extract (5 μg/ml), Coleus forskohlii extract (5 μg/ml), and the combination of both Cassia occidentalis extract (5 μg/ml) and Coleus forskohlii extract (5 μg/ml).

FIGS. 7 to 10 depict a subject with vitiligo before (FIGS. 7 and 9) and after (FIGS. 8 and 10) topical treatment with a composition of the present invention during 30 days.

### DETAILED DESCRIPTION OF EXAMPLES

The application of the composition on the skin of patients afflicted by skin disease related to depigmentation, such as vitiligo, (for example once a day, or two daily applications) has shown response featuring the reduction of depigmentation and reduction in the progression of disease, and even partial repigmentation of white patches. In another aspect of the present invention, the use of the combination of these two vegetal active extracts under a cosmeceutically acceptable form stimulates the survival, proliferation and differentiation of melanocytes.

### Example 1—Effect of the Composition According to the Invention Comprising Coleus forskohlii Extract and Cassia occidentalis Extract or Cassia alata Extract on Melanocytes Proliferation

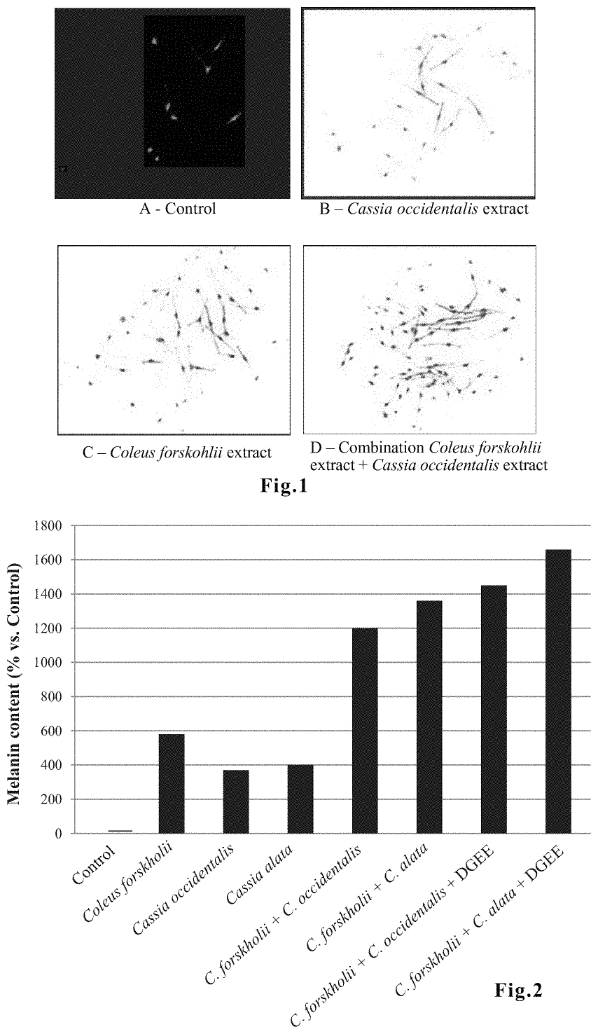

FIG. 1 depicts culture of human melanocytes treated with control, with each of the extracts, and with the combination of both extracts for 96 hours. Compared to control, Coleus forskohlii extract and Cassia occidentalis extract show increased melanocyte proliferation, and combination of Coleus forskohlii extract and Cassia occidentalis extract show increased melanocyte proliferation, more important than with each active ingredient taken separately.

### Example 2—Effect of the Composition According to the Invention Comprising Coleus forskohlii Extract and Cassia occidentalis Extract on Melanogenesis

FIG. 2 depicts melanogenesis in B16 melanoma murine melanocytes treated with control, with Coleus forskohlii extract, with Cassia occidentalis extract, with Cassia alata extract and with the combination of Coleus forskohlii extract+Cassia occidentalis extract or Coleus forskohlii extract+Cassia alata extract, ±diethylene glycol monoethyl ether. It is obvious that the combination of Coleus forskholii with Cassia occidentalis or Cassia alata stimulates melanogenesis in a more important manner than each extract taken separately, compared with control. Moreover, it is illustrated that both plant extracts (Coleus forskholii+Cassia occidentalis or Coleus forskholii+Cassia alata) act synergetically to increase the melanin synthesis. Besides, FIG. 2 demonstrates that the addition of diethylene glycol monoethyl ether (DGEE) improves the melanogenesis induced by the combination of Coleus forskholii+Cassia occidentalis or Coleus forskholii+Cassia alata.

### Example 3—Effect of the Composition According to the Invention Comprising Coleus forskohlii Extract and Cassia occidentalis Extract on the Expression of Molecular Factors Melanogenesis

Melanogenesis is a complex biological process where eumelanin and phaeomelanin derive from a common tyrosinase-dependent pathway with the same precursor, tyrosine. From dopaquinone, the eumelanin and phaeomelanin pathways diverge. Two enzymes crucial to eumelanogenesis are the tyrosinase-related proteins TRP1 (also known as GP75 or b-locus) and TRP2 (also known as dopachrome tautomerase, DCT).

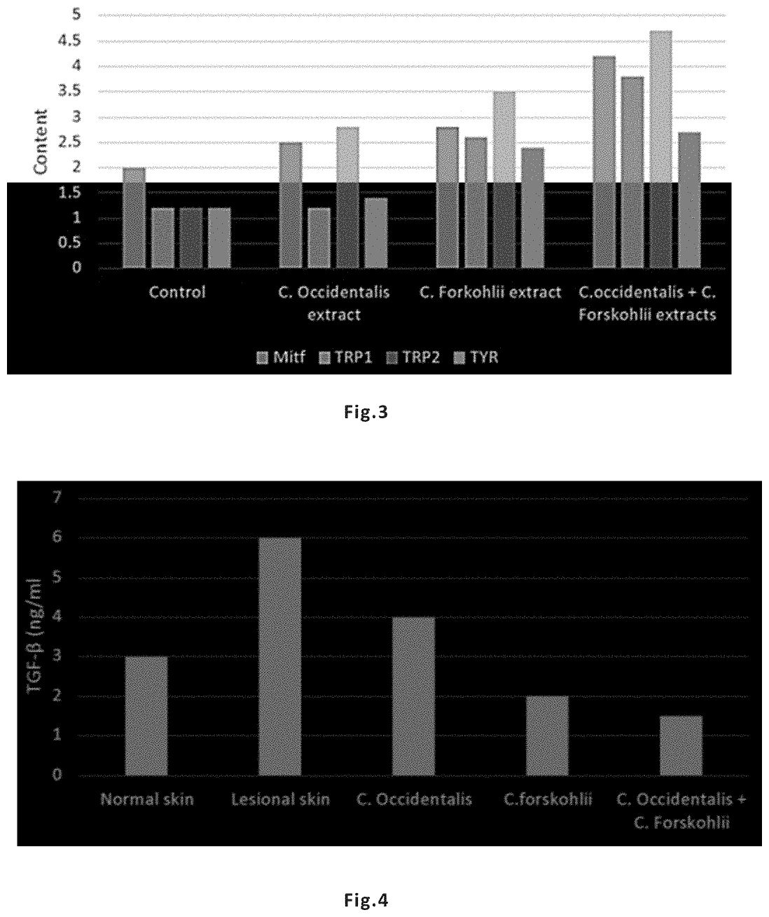

Microphthalmia-associated transcription factor (Mitf) is involved in lineage-specific pathway regulation of melanocytes. All these factors are of utmost importance for the eumelanogenesis. As depicted in FIG. 3, the Applicant have discovered that Cassia occidentalis extract, as well as Coleus forskohlii extract, are able to increase the production of all four factors in A375 melanocytes. Unexpectedly, the combination of Cassia occidentalis extract with Coleus forskohlii extract is able to increase the production of all four aforementioned factors in a more important manner than each extract taken separately.

### Example 4—Effect of the Composition According to the Invention Comprising Coleus forskohlii Extract and Cassia occidentalis Extract on the Expression of TGF-β

It is known that the levels of Transforming Growth Factor-β are usually increased in the skin of patients with vitiligo. In non-segmental vitiligo, the body surface area of lesions is positively correlated with elevated TGF-β1. Significant increase in the expression of TGF-β was observed in active patients with vitiligo, whereas no change was observed in stable patients, suggesting that high levels of TGF-β promote the progression of the disease. As shown in FIG. 4, the Applicant have been able to confirm these findings in skin biopsies of normal skin and lesional skin of patients with vitiligo. They have also observed that treatment of these skin explants with Cassia occidentalis extract was reducing the levels of TGF-β, but at a lesser extent than Coleus forskohlii, and unexpectedly they discovered that the combination of Cassia occidentalis extract with Coleus forskohlii extract was able to reduce the skin levels of TGF-β at a much higher extent that each of these two extracts taken separately.

### Example 5—Effect of the Composition According to the Invention Comprising Coleus forskohlii Extract and Cassia occidentalis Extract on the Expression of TNF-α

Levels of TNF-α were also shown to be increased in skin biopsies of vitiligo lesions compared to normal skin. Cases of refractory generalized vitiligo showed high tissue levels of TNF-α. Considering these cases, patients with a strong TNF-α staining were characterized by a higher vitiligo disease activity score than patients with a weak staining, which suggests a probable role of TNF-α in the pathogenesis of vitiligo.

A study revealed significant increase in TNF-α transcript and protein levels in vitiligo patients compared to controls. It is commonly thought that TNF-α can inhibit melanogenesis by decreasing the intracellular levels of tyrosinase and tyrosinase-related protein 1, involved in both melanogenesis and prevention of melanocyte death. TNF-α-treated melanocytes show marked cellular shrinking and reduced melanin production in vitro, as well as downregulation of Mitf, a transcription factor essential in the regulation of melanocyte development, proliferation, death, and melanogenesis.

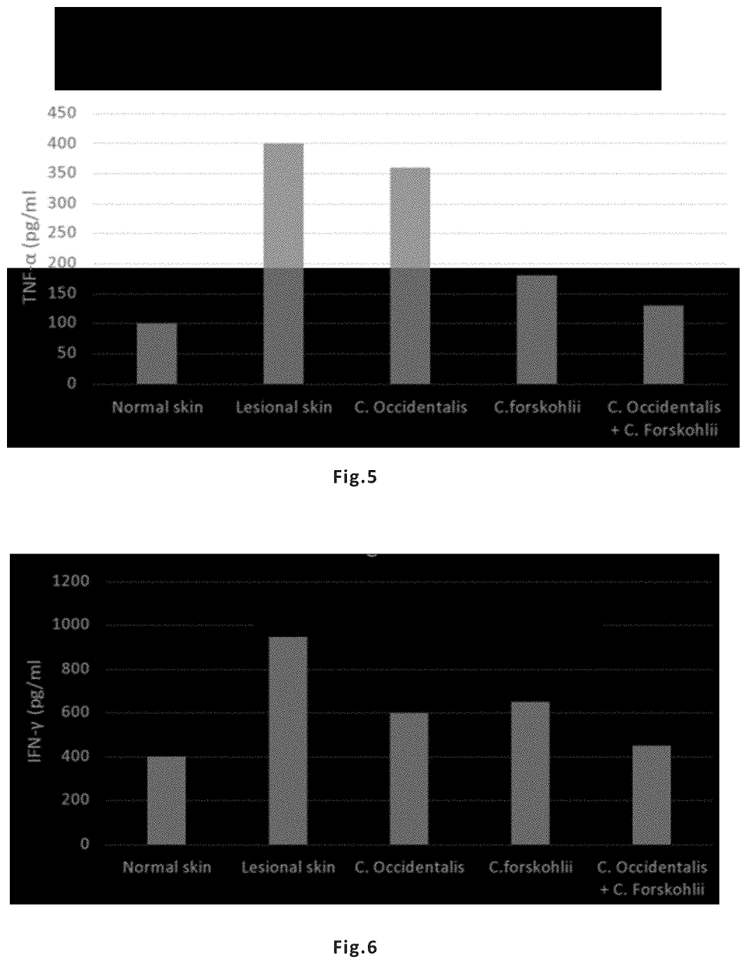

As depicted in FIG. 5, the Applicant have been able to confirm higher levels of TNF-α in biopsies taken from lesional skin of vitiligo patients, compared to normal skin of the same patients. Further, treating these explants with Cassia occidentalis extract, these levels of TNF-α were slightly decreased. The reduction was much more important when treated with Coleus forskohlii extract, and unexpectedly the Applicant discovered that treatment with the combination of Cassia occidentalis extract and Coleus forskohlii extract was leading to a decrease in TNF-α much more substantial than the decrease observed with each of these extracts taken separately.

### Example 6—Effect of the Composition According to the Invention Comprising Coleus forskohlii Extract and Cassia occidentalis Extract on the Expression of IFN-γ

It is well recognized that increased expression of the cytokine interferon-γ (IFN-γ) plays a pivotal role in vitiligo-induced depigmentation. It was demonstrated that IFN-γ inhibits melanogenesis in primary cultured human melanocytes by altering melanogenic enzyme mRNA expression and, more importantly, that IFN-γ directly induces melanocyte apoptosis. It is suggested that IFN-γ inhibits both basal and α-MSH-induced melanogenesis by inhibiting MITF expression and that IFN-γ plays a role in controlling inflammation- or UV-induced pigmentary changes. The Applicant have also demonstrated in skin explants from lesional skin of vitiligo patients increased levels of IFN-γ compared with normal skin of the same patients. As depicted in FIG. 6, treating these explants with Cassia occidentalis extract leaded to a decrease in the level of IFN-γ more or less similar to that produced by treatment with Coleus forskohlii extract. Unexpectedly the Applicant found that the association of both extracts leaded to a higher reduction of IFN-γ levels.

### Example 7—Effect of the Composition According to the Invention Comprising Coleus forskohlii Extract and Cassia occidentalis Extract to Treat Vitiligo

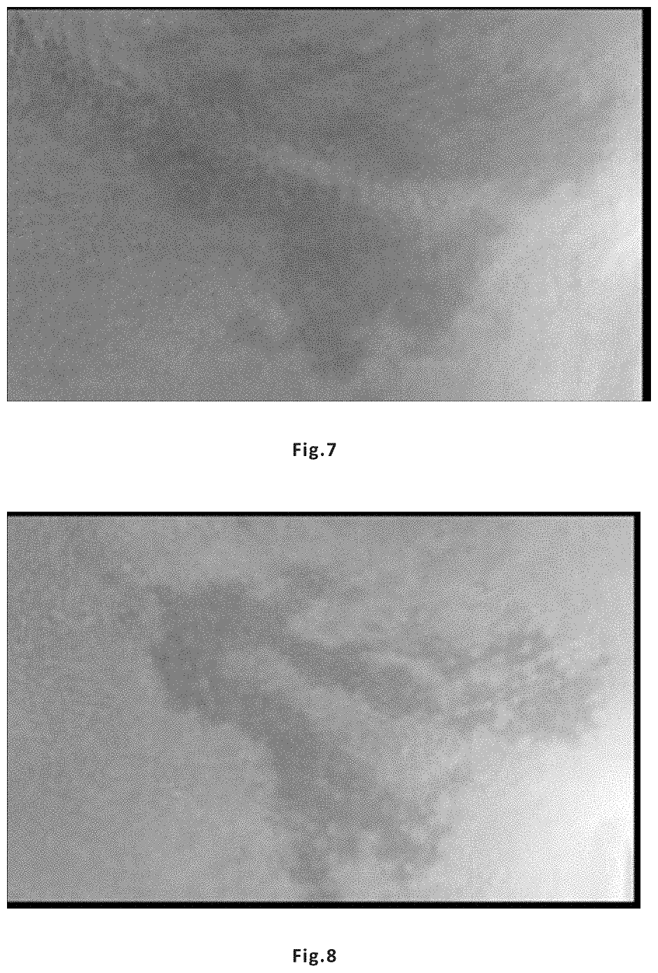

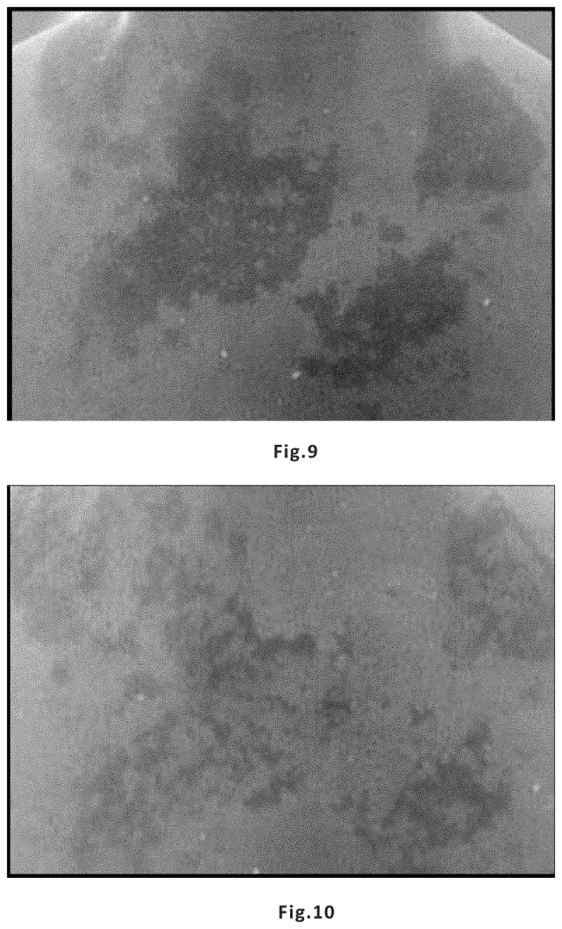

FIGS. 7 to 10 depict the results of increased melanogenesis and melanocyte proliferation four weeks after topical application once a day of the composition of the invention to two subjects with vitiligo.

The treatment of vitiligo with a composition comprising a combination of Coleus forskohlii extract, Cassia occidentalis extract and/or Cassia alata extract in a cosmeceutically acceptable vehicle containing also a penetration enhancer, and whose pH is maintained as close as possible to 6.8 was found to be successful.

### Example 8—Comparative Study of the Composition According to the Invention with Other Compositions

8.1—Composition According to the Invention

At ambient temperature, and under light stirring, in 300 g (30%) of distilled water, are added respectively:
3.5 g (0.35%) of a gelling agent featuring a polyvinyl carboxy polymer crosslinked with ethers of pentaerythritol, known under the commercial brand Carbomer 940.
80 g (8%) of an extract of Coleus forskohlii obtained by the extraction of dried roots of the plant by a blend of ethanol and propylene glycol.
50 g (5%) of an extract of Cassia occidentalis and/or Cassia alata obtained by the extraction of dried pods or leaves of these plants by a methanolic solution.
10 g (1%) of a liquid preservative agent named phenoxyethanol.
1 g (0.1%) of an ion chelator known as disodium EDTA.
15 g (1.5%) of a penetration enhancer known under the name of diethylene glycol monoethyl ether.

One stirs slowly at ambient temperature, until a homogenous mixture is obtained.

Separately, under rapid stirring (1′000 rpm) and at 80° C. are mixed in this order:
10 g (1%) of an excipient made of cetyl alcohol.
90 g (9%) of another excipient made of propylene glycol dipelargonate.
80 g (8%) of another excipient made of decyl oleate.
80 g (8%) of another excipient made of isostearyl isosterate.

Under rapid stirring, a perfect homogenisation can be obtained.

Then, distilled water heated to 82° C. is added in the above mixture in a quantity of 280.5 g (28.05%) under rapid stirring.

When the emulsion is constituted, and temperature back to 30-35° C. is added the first phase containing the vegetal active ingredients and then are added 6 grams (0.6%) of a 50% solution of triethanolamine in order to adjust the pH.

Slow stirring is continued still reaching the temperature of 25° C.

This composition is an oil-in-water emulsion, the colour is beige, it is smooth, unctuous, semi-liquid and its pH is ranging from 6.5 to 7.1 Its odour is sui generi.

This preparation is stable over time and may be applied on the skin once or twice daily during an unlimited period of time. One can observe, sometimes as soon as ten days, more generally in the course of the month following its application, a limitation and even a stop in depigmentation and usually a visible repigmentation as seen in FIGS. 7 to 10.

Further applying this emulsion prevents further depigmentation.

8.2—Composition Comprising Solely Coleus forskohlii Extract

Example 1 is repeated incorporating in the emulsion solely Coleus forskohlii extract. The effect is more limited and appears after a longer time.

8.3—Composition Comprising Solely Cassia occidentalis Extract

Example 1 is repeated incorporating in the emulsion solely Cassia occidentalis extract. The effect is very limited and appears after a longer time.

8.4—Composition Comprising Coleus forskohlii Extract and Cassia occidentalis Extract

Example 1 is repeated without incorporation of diethylene glycol monoethyl ether. The emulsion obtained has a pretty similar appearance and the same physical properties than in example 1. However, a limitation of depigmentation is observed in a slower and more sporadic manner than in example 1.

8.5—Composition According to the Invention with an Acidic pH

Example 1 is repeated without adjusting the pH between 6.5 to 7.1, but with a pH value more acidic, i.e. between 5.5 and 6.5. The emulsion obtained has a pretty similar appearance and the same physical properties than in example 1 (except for its pH).

However, a limitation of depigmentation is observed in a slower and more sporadic manner than in example 1.

8.6—Composition without any Plant Extracts According to the Invention

Example 1 is repeated replacing the vegetal extracts of Coleus forskohlii and Cassia occidentalis by other vegetal extracts. No result is observed.

These examples perfectly demonstrate the activity related to the synergistic effect resulting from the combination in the composition of Coleus forskohlii extract with Cassia occidentalis and/or Cassia alata extract with diethylene glycol monoethyl ether to enhance the penetration, and with a pH as close as possible to 6.8 on the limitation of depigmentation and the reduction of the inflammatory front in the course of the process of development of vitiligo and its further repigmentation.

1. A topical composition comprising:
a) a cosmeceutically acceptable vehicle;
b) an extract of Coleus forskohlii; and
c) an extract of Cassia occidentalis and/or Cassia alata.

2. A topical composition according to claim 1, characterized in that it further comprises a penetration enhancer.

3. A topical composition according to claim 1, wherein the extract of Coleus forskohlii is a root extract.

4. A topical composition according to claim 1, wherein the extract of Cassia occidentalis is a pod extract.

5. A topical composition according to claim 1, wherein the extract of Cassia alata is a leave extract.

6. A topical composition according to claim 1, wherein the weight ratio of Coleus forskohlii extract:Cassia occidentalis and/or Cassia alata represents 3:1.

7. A topical composition according to claim 1, wherein:
the Coleus forskohlii extract represents between 5.0 and 8.0% in weight of the composition; and
the Cassia occidentalis and/or Cassia alata extract represents between 2.0 and 5.0% in weight of the composition.

8. A topical composition according to claim 1, wherein the penetration enhancer is diethylene glycol monoethyl ether and represents between 0.5 and 5.0% in weight of the composition, advantageously between 1.0 to 2.0%.

9. A topical composition according to claim 1, wherein the pH is ranging between 6.5 to 7.1.

10. A topical composition according to claim 1, wherein said composition is formulated as a leave-on product or a rinse-off product.

11. A topical composition according to claim 1, for use to promote the proliferation of melanocytes in the skin and/or in the hair follicle unit.

12. A topical composition according to claim 1, for use to promote the melanogenesis in the skin and/or in the hair follicle unit.

13. A topical composition according to claim 1, for use to treat canitie.

14. A topical composition according to claim 1, for use as a tanning agent.

15. A topical composition according to claim 1, for use to treat skin disease related to depigmentation in a human subject.

16. A topical composition for use according to claim 15, wherein skin disease related to depigmentation is selected from the group consisting of pytiriasis alba, pytiriasis versicolor, idiopathic guttate hypomelanosis, progressive macular hypomelanosis, post-inflammatory hypopigmentation and vitiligo.

17. A topical composition for use according to claim 15, wherein skin disease related to depigmentation is vitiligo.
